(19) United States
(12) Patent Application Publication
(10) Pub. No.: US 2017/0340865 A9
(43) Pub. Date: Nov. 30, 2017

(54) PROLATE SPHEROID-SHAPED BALLOON WITH CENTRAL HINGE

(71) Applicant: TRANSLATIONAL BIOLOGIC INFUSION CATHETER, LLC, Gilbert, AZ (US)

(72) Inventors: Nabil Dib, Paradise Valley, AZ (US); Alan Iranian, Maple Grove, MN (US)

(21) Appl. No.: 15/248,373

(22) Filed: Aug. 26, 2016

Related U.S. Application Data
(63) Continuation-in-part of application No. 14/201,495, filed on Mar. 7, 2014, now abandoned.

(51) Int. Cl. A61M 25/10

(52) U.S. Cl. CPC A61M 25/1002; A61M 25/104; A61M 25/10181; A61M 2207/00; A61M 2210/12; A61M 2025/1068

(57) ABSTRACT

An elongated, tubular-shaped balloon for a balloon catheter includes three regions along its length, in sequence: a proximal region, an intermediate region, and a distal region. The intermediate region is defined by a curved outer surface that is established by a radius of curvature r₁. Similarly, a curved inner surface for the intermediate region is established by a radius of curvature r₂, wherein r₁≧r₂. Balloon thickness at the center of the intermediate region is t_{c}, while balloon thickness in both the proximal and distal regions is t (t>t_{c}). The stretchability and bendability of the balloon material is directly proportional to the thickness of the balloon, to thereby shape the balloon as a prolate spheroid when inflated.

This application is a continuation-in-part of application Ser. No. 14/201,495, filed Mar. 7, 2014, which is currently pending. The contents of application Ser. No. 14/201,495 are incorporated herein by reference.

### FIELD OF THE INVENTION

The present invention pertains generally to catheters having an inflatable balloon that can be used to position the distal end of the catheter at a target site in the vasculature of a patient. More particularly, the present invention pertains to a balloon for a balloon catheter that provides minimal radial forces between the balloon and a vessel wall when inflated to decrease the incidence of vessel dissection and perforation. The present invention is particularly, but not exclusively, useful as a balloon that can adapt to different vessel diameters to minimize the need for multiple balloon catheters.

### BACKGROUND OF THE INVENTION

Inflatable balloons are often used to dilate a blockage in an artery with minimal radial forces on the arterial wall. This is done to cause less vascular injury such as dissection and perforation. Also, balloons can be employed for placing stents in the vasculature of a patient. In another application, balloons can be used to anchor a portion of a catheter at a target site in the vasculature of a patient. Typically, for this purpose, an inflatable balloon is mounted at the distal end of the catheter. The distal end of the catheter is then inserted into the patient and advanced within the patient's vasculature to a treatment site. There, at the treatment site, the balloon is inflated until it contacts the wall of the vessel. Once positioned, the catheter can be used, for example, to perform diagnostic imaging, infusion of a medicament, the placement of a stent, or to anchor the catheter as required by a particular protocol,

Generally, for these procedures, balloons are made of a compliant material. In more detail, balloons made of a compliant material continue to expand as the internal pressure in the balloon is increased. This is to be contrasted with a non-compliant balloon which expands to a predetermined size and shape as the internal pressure in the balloon is increased. In one application, a non-compliant balloon can be used to exert force on a vessel wall, for example, to expand a constricted artery.

Heretofore, compliant balloons have been used which, when inflated, establish a substantially tubular, ‘hot dog’ shape within a vessel. With increasing inflation, the hot dog shaped balloons elongate, increasing the contact area between the balloon and the internal wall of the vessel. This results in a substantial contact area between the balloon and internal vessel wall. In some cases, however, a substantial contact area between the balloon and internal vessel wall is undesirable. Moreover, it may be undesirable to have a balloon/vessel wall contact area that varies with inflation pressure.

In light of the above, it is an object of the present invention to provide a balloon for a catheter that can operationally adapt to different vessel diameters and tolerate high-pressure inflation within the vasculature of a patient. Another object of the present invention is to provide a balloon for a catheter that maintains a substantially constant inter-contact surface area between the balloon and a vessel wall over a range of inflation pressures. Yet another object of the present invention is to provide a prolate spheroid-shaped balloon that is easy to use, is simple to implement and is comparatively cost effective.

### SUMMARY OF THE INVENTION

In accordance with the present invention, a balloon system for positioning a distal end of a catheter at a treatment site includes an elongated catheter shaft that is formed with a lumen. For the balloon system, the shaft defines a longitudinal axis, extends from a proximal end to a distal end, and has an outer diameter dₒ.

In addition to the shaft, the system includes a tubular shaped balloon membrane that is made of a compliant material such as urethane. Typically, the balloon membrane has a length L between its proximal end and its distal end. In any event, the actual value for the length L is discretionary and will depend on the particular application. For the system, the proximal and distal ends of the balloon membrane are affixed to an outer surface of the shaft to establish an inflation chamber between the balloon membrane and the outer surface of the shaft.

For the present invention, the balloon membrane can have a non-uniform thickness between the proximal and distal ends of the membrane to establish a selected membrane shape when the balloon is inflated. For example, the selected membrane shape can be a prolate spheroid.

In one embodiment of the balloon system, the balloon membrane can be thicker at the ends (i.e. the proximal and distal ends) than a region midway between the ends. With this arrangement, a relatively short and a relatively flat inter-contact surface in the midway region of the membrane is obtained when the balloon is inflated. In more detail, the balloon membrane can have a central thickness t_{c} in the region midway between the proximal and distal membrane ends and a membrane thickness tₑ at the proximal and distal membrane ends, with tₑ>t_{c}.

Also for the balloon system, an inflation unit is included to inflate the balloon. For example, an inflation lumen can be formed in the catheter shaft to establish fluid communication between the inflation unit and the inflation chamber of the balloon.

During an inflation of the balloon by an inflation pressure Pᵢ, a radial distance r_{c} is established from the outer surface of the shaft to the inter-contact surface of the midway region. In addition, for the balloon system of the present invention, the radial distance r_{c} varies proportionally with changes in Pᵢ inside the inflation chamber. Typically, the radial distance r_{c} will be as required by the application. For example, it will usually be less than about 35 mm with a balloon inflation pressure Pᵢ less than about 15 atmospheres. In one embodiment, a balloon is designed to be inflated up to 14 atm of pressure.

In one aspect of the present invention, the balloon membrane is designed such that sequential configurations of the balloon membrane during an inflation cycle present a substantially same area for the inter-contact surface of the midway region. For example, this functionality can be achieved by controlling the thickness between the proximal and distal ends of the membrane during the balloon membrane manufacturing process.

For another perspective of the present invention, the elongated balloon membrane can be considered as having three distinguishable regions along its length L. These are, in sequence: a proximal region, an intermediate region, and a distal region. As envisioned for the present invention, the balloon will be made of a compliant material. Moreover, in the intermediate region, the balloon membrane will be thinner than it is in the proximal and distal regions of the balloon. Consequently, the balloon membrane in the intermediate region will be more stretchable and bendable than it is in the proximal and distal regions.

Structurally, the intermediate region is defined by a curved outer surface that is established by a radius of curvature r₁. It also has a curved inner surface that is established by a radius of curvature r₂. For the present invention, r₂≦r₁.

The extent of the intermediate region between the proximal and the distal regions of the balloon can be varied from balloon to balloon, as needed. More specifically, with the intermediate region always centered midway between the ends of the balloon, the intermediate region can be extended from the midway point to cover as little as 10% of the balloon's length L, or as much as 90% of the length L.

It is an important feature of the balloon for the present invention that its thickness at the center of the intermediate region has a predetermined value, t_{c}. On the other hand, the thickness of the balloon membrane in both the proximal and distal regions is greater than or equal to a value t. In this combination, t will always be greater that t_{c} (t>t_{c}). With this in mind, the stretchability and bendability within the different regions of the balloon will vary for two reasons. First, these functional characteristics are dependent on the material that is used to manufacture the balloon. Second, they are directly proportional to the thickness of the balloon material. Consequently, depending on selected dimensions for balloon thickness in the different regions, an inflated balloon will assume customized configurations for its generalized shape as a prolate spheroid.

It is to be noted that the present invention anticipates the creation of discontinuities on the inner surface of the balloon during manufacture. In particular, it is anticipated that discontinuities will occur at the interfaces between the intermediate region and the proximal and distal regions, respectively. Specifically, a discontinuity is to be expected for designs wherein the thickness t_{c} remains constant throughout the intermediate region. Also, a discontinuity will occur whenever the separation between the surfaces generated by r₁ are r² at the interface is less than t. For such discontinuities, and others when experienced, the present invention envisions the creation of minimally intrusive transition zones for smoothing the discontinuities.

### BRIEF DESCRIPTION OF THE DRAVVINGS

The novel features of this invention, as well as the invention itself, both as to its structure and its operation, will be best understood from the accompanying drawings, taken in conjunction with the accompanying description, in which similar reference characters refer to similar parts, and in which:

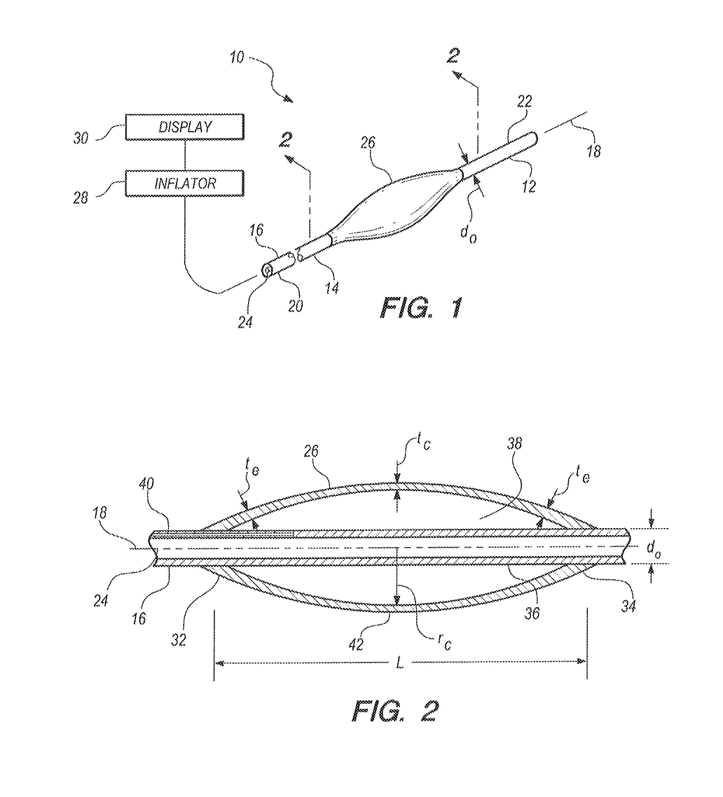

FIG. 1 is a schematic/perspective view of the balloon system of the present invention;

FIG. 2 is a cross-section view of a portion of the balloon system as seen along the line 2-2 in FIG. 1, shown with the balloon inflated by an inflation pressure Pᵢ;

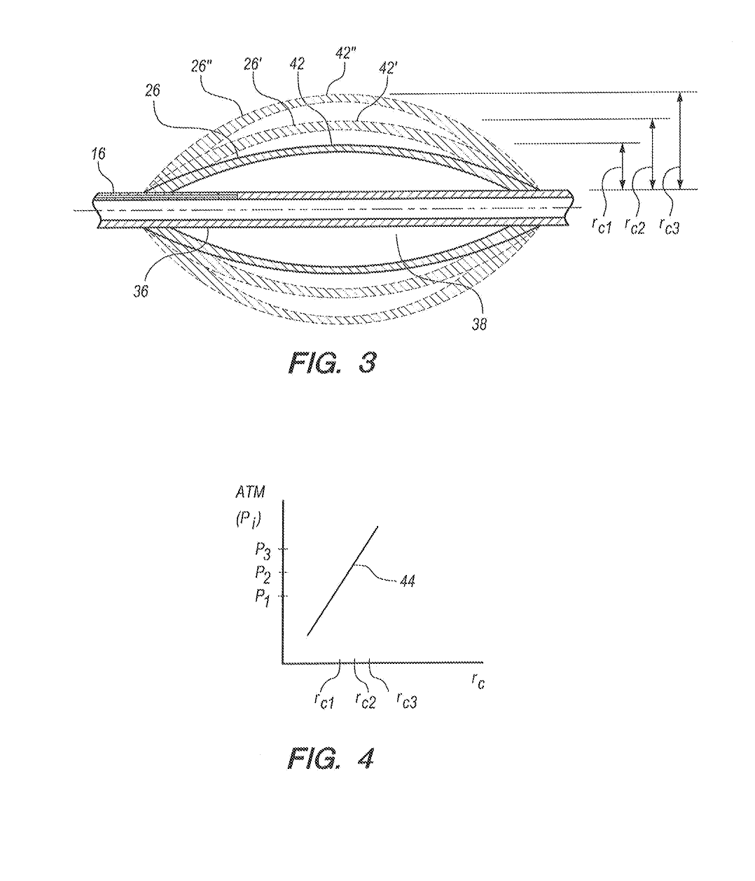

FIG. 3 is a cross-section view of a portion of the balloon system as seen along the line 2-2 in FIG. 1, shown with the balloon inflated by an inflation pressure Pᵢ together with two other balloon configurations (shown by dotted lines) corresponding to two other inflation pressures;

FIG. 4 is graph showing a balloon inflation pressure (ordinate) as a function of radial distance r_{c} from the outer surface of the shaft to the inter-contact surface of the midway region (abscissa);

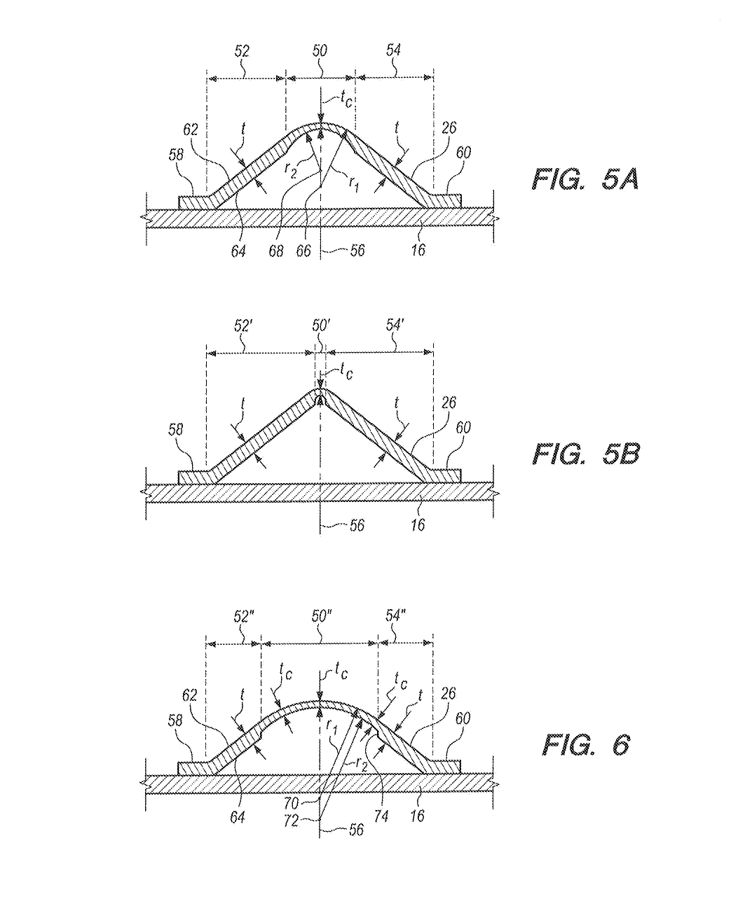

FIG. 5A is a cross-section view of a portion of the balloon system, as seen along the line 2-2 in FIG. 1, showing the incorporation of curved surfaces with different radii of curvature, to create regions of the balloon having different stretchability and bendability structural capabilities;

FIG. 5B is a cross-section view of the portion of the balloon system shown in FIG. 5A, wherein the extent of respective regions have been changed along the length of the balloon; and

FIG. 6 is a cross-section view of the portion of the balloon system shown in FIG. 5A, showing a region wherein the curved surfaces have the same radius of curvature.

### DESCRIPTION OF THE PREFERRED EMBODIMENTS

Referring initially to FIG. 1 a balloon system in accordance with the present invention is shown and is generally designated 10. In one application, the balloon system 10 can be used to position a distal end 12 of a catheter 14 at a treatment site within the vasculature of a patient (not shown). FIG. 1 also shows that the balloon system 10 includes a shaft 16 that defines a longitudinal axis 18, extends from a proximal end 20 to a distal end 22, and has an outer diameter dₒ. FIG. 1 also shows that the shaft 16 is formed with a lumen 24.

Continuing with FIG. 1, it can be seen that the balloon system 10 also includes a tubular shaped balloon membrane 26. Typically, for the present invention, the balloon membrane 26 is made of a compliant material such as urethane. FIG. 1 also shows that the balloon system 10 can include an inflator 28 that is operationally connected to the proximal end 20 of the shaft 16 to selectively inflate the balloon. Also, as shown, a display 30 can be operationally connected to the inflator 28 to provide information, such as inflation pressure, to a user (not shown), such as a physician, during a balloon inflation.

FIG. 2 shows that the balloon membrane 26 has a length L. between its proximal end 32 and its distal end 34 and, typically, L will be between about 8-35 mm for use in the coronary and between about 20-150 mm for use in the peripheral arteries. It can also be seen in FIG. 2 that the proximal end 32 and distal end 34 of the balloon membrane 26 are affixed to an outer surface 36 of the shaft 16. With this cooperative structural arrangement, an inflation chamber 38 is established between the balloon membrane 26 and the outer surface 36 of the shaft 16. Also, FIG. 2 shows that the shaft 16 can be formed with an inflation lumen 40 to establish fluid communication between the inflator 28 (see FIG. 1) and the inflation chamber 38.

Continuing with reference to FIG. 2, it can be seen that the balloon membrane 26 can be thicker at the ends (i.e. the proximal end 32 and distal end 34) than a region 42 that is midway between the proximal end 32 and distal end 34. As shown, the balloon membrane 26 can have a central thickness t_{c} in the region 42 midway between the proximal end 32 and distal end 34 and a membrane thickness tₑ at the proximal end 32 and distal end 34, with tₑ>t_{c}. This arrangement allows for a relatively short and a relatively flat inter-contact surface in the midway region 42 of the membrane 26 to be obtained when the balloon is inflated. FIG. 2 illustrates that the balloon membrane 26 can have a non-uniform thickness between the proximal end 32 and distal end 34 to establish a selected membrane shape when the balloon is inflated. For the embodiment shown in FIG. 2, the selected membrane shape is a prolate spheroid. FIG. 2 shows the balloon inflated to an inflation pressure Pᵢ. As shown, at the inflation pressure Pᵢ, the midway region 42 of the membrane 26 is spaced at a radial distance r_{c} from the axis 18 of the shaft 16.

FIGS. 3 and 4 illustrate that the radial distance between the midway region 42 of the membrane 26 and the outer surface 36 of the shaft 16 varies proportionally with changes in Pᵢ inside the inflation chamber 38. Specifically, FIG. 3 shows the membrane 26 at an inflation pressure P₁ has a radial distance r_{c1} between the midway region 42 of the membrane 26 and the outer surface 36 of the shaft 16. At an inflation pressure P₂, with P₂>P₁, membrane 26′ has a radial distance r_{c2}, with r_{c2}>r_{c1}, between the midway region 42′ of the membrane 26′ and the outer surface 36 of the shaft 16. Also, at an inflation pressure P₃, with P₃>P₂, membrane 26″ has a radial distance r_{c3}, with r_{c3}>r_{c2}, between the midway region 42″ of the membrane 26″ and the outer surface 36 of the shaft 16. FIG. 3 also illustrates that the balloon membrane 26 is designed such that sequential configurations of the balloon membrane 26 during an inflation cycle present a substantially same area for the inter-contact surface of the midway region 42. FIG. 4 shows a plot 44 of balloon inflation pressure (ordinate) as a function of radial distance r_{c} from the outer surface 36 (FIG. 3) of the shaft 16 to the inter-contact surface of the midway region 42 (abscissa). From FIG. 4, it can be seen that the radial distance rc between the midway region 42 (FIG. 3) of the membrane 26 and the axis 18 of the shaft 16 varies proportionally with changes in Pᵢ inside the inflation chamber 38.

In another aspect of the present invention, balloon membrane 26 can be constructed with separate, identifiable and distinguishable regions. Specifically, as shown in FIG. 5A, the balloon membrane 26 can be manufactured with an intermediate region 50 that is positioned between a proximal region 52 and a distal region 54. FIG. 5A also shows that with a line 56, drawn perpendicular to the shaft 16 and centered between the proximal end 58 and the distal end 60 of the balloon membrane 26, the balloon membrane 26 will have a thickness t_{c} along the line 56. Stated differently, t_{c} is the thickness of the membrane 26 between the outer surface 62 and the inner surface 64 at the midpoint of the intermediate region 50.

It is an important feature of the present invention that the extent of the intermediate region 50 is determined by a configuration of the outer surface 62 of the balloon membrane 26. Specifically, in the intermediate region 50, the outer surface 62 will conform to a curve having a radius of curvature r₁ around a point 66 on line 56. Further, in the intermediate region 50, the inner surface 64 of balloon membrane 26 will conform to a curve having a radius of curvature r₂ around a point 68 on the line 56. Also, as shown in FIG. 5A, the balloon membrane 26 will have a thickness t in both the proximal region 52 and the distal region 54. Typically, the thickness t will be constant, and it will be the same, for both the proximal region 52 and the distal region 54. As envisioned for the present invention t_{c} will always be less than t (t_{c}<t), and r₁ will be greater than or equal to r₂ (r₁≧r₂).

With the above in mind, and depending on the linear extent of the intermediate region 50 within the length L of the balloon membrane 26, several different variations for configurations of the present invention are possible, For one variation, as shown in FIG. 5B, the intermediate region 50′ can be diminished to around 5% of L. For this variation, the proximal region 52′ and the distal region 54′ are appropriately expanded to maintain the intermediate region 50′ centered for the balloon membrane 26. In this case, like the above disclosure for FIG. 5A, t_{c} will still be less than t (t_{c}<t), and r₁ will still be greater than or equal to r₂ (r₁≧r₂). For the variation shown in FIG. 5B, the intermediate region 50 will function fundamentally as a so-called “living hinge”.

For another variation in the configuration of the balloon membrane 26, FIG. 6 shows that t_{c} can be held constant throughout the intermediate region 50″. In this case, t_{c} will still be less than t (t_{c}< and ≠t), but r₁ will equal r₂ (r₁=r₂). In this variation, the present invention envisions the outer surface 62 will conform to a curve in the intermediate region 50″ having a radius of curvature r₁ around a point 70 on line 56, while the inner surface 64 of balloon membrane 26 will conform to a curve having a radius of curvature r₂ around a point 72 on the line 56.

It is to be appreciated that the proximal region 52 (FIG. 5A) and the proximal region 52″ (FIG. 6), as well as the distal region 54 (FIG. 5A) and the distal region 54″ (FIG. 6) are variable, depending on the extent of intermediate region 50 (Fig, 5A) or intermediate region 50″ (FIG. 6). Specifically, as envisioned for the present invention, the extent of intermediate region 50, for variations shown in FIG. 5A and FIG. 6, can be selected to be anywhere in a range between 90% of L and 5% of L. Further, in the variations for the present invention shown in FIG. 5A and FIG. 6, the present invention anticipates the creation of a discontinuity 74 (best shown in FIG. 6) at the interface between the intermediate region 50 and the regions 52 and 54. As intended for the present invention, if necessary, the discontinuity 74 can be modified to present a smooth transition on the inner surface 64.

In accordance with the above disclosure, it is also to be appreciated that when the regions 50, 52 and 54 are respectively rotated around the axis 18, they will, in combination, form a prolate spheroid. In detail, the intermediate region 50 will be formed as an annulus, and both the proximal region 52 and the distal region 54 will respectively be formed conical surfaces.

While the particular prolate spheroid-shaped balloon with central hinge as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system which comprises:
an elongated shaft formed with a lumen, wherein the shaft defines a longitudinal axis, has a proximal end and a distal end, and has an outer surface;
a tubular shaped balloon membrane having a proximal end affixed to an outer surface of the shaft and a distal end affixed to the outer surface of the shaft to establish an inflation chamber between the balloon membrane and the outer surface of the shaft, wherein the balloon membrane has a central thickness t_{c} at the middle of an intermediate region midway between the proximal and distal ends of the membrane, wherein the intermediate region of the membrane is defined by a first arc on an outer surface of the membrane and includes a second arc on an inner surface of the membrane, wherein the first arc and the second arc are coplanar with the longitudinal axis of the shaft, wherein the first arc has a radius of curvature r₁ around a first point on a line perpendicular to the shaft and the second arc has a radius of curvature r₂ around a second point on the line, wherein the line intersects the shaft at a midpoint between the proximal end and the distal end of the membrane, and wherein r₁≧r₂ and the intermediate region of the membrane has a thickness t_{c} along the line; and
an inflation unit connected in fluid communication with the inflation chamber of the balloon to inflate the balloon.

2. The system recited in claim 1 wherein the membrane has a proximal region and a distal region, with the intermediate region positioned therebetween.

3. The system recited in claim 2 wherein the balloon membrane is made of a compliant material.

4. The system recited in claim 3 wherein the stretchability and bendability of the membrane material is directly proportioned to the thickness of the membrane.

5. The system recited in claim 3 wherein the distal region and the proximal region have a same, constant thickness t, and t is greater than t_{c}.

6. The system recited in claim 3 wherein the balloon membrane has a length L between the proximal and the distal end, and L is less than 150 mm.

7. The system recited in claim 6 wherein the intermediate region corresponds with less than 30% of L.

8. The system recited in claim 3 wherein the intermediate region corresponds with less than 90% of L.

9. The system recited in claim 3 wherein the intermediate region corresponds to 5% of L.

10. The system recited in claim 1 wherein during an inflation of the balloon, a radial distance r_{c} along the line from the longitudinal axis of the shaft to the outer surface of the intermediate region, is established by an inflation pressure Pᵢ inside the inflation chamber, wherein r_{c} varies proportionally with changes in Pᵢ inside the inflation chamber, and wherein the radial distance r_{c} is less than 35 mm.

11. The system recited in claim 3 wherein the compliant material is urethane and the inflation pressure Pᵢ is less than about 15 atmospheres.

12. A method for manufacturing a balloon catheter which comprises the steps of:
providing an elongated shaft formed with a lumen, wherein the shaft defines a longitudinal axis and has a proximal end and a distal end;
differentiating regions of a tubular-shaped balloon membrane between a proximal end of the balloon and a distal end of the balloon, wherein the regions are designated as a proximal region, a distal region and an intermediate region therebetween, wherein the balloon membrane has an outer surface and an inner surface;
forming the outer surface of the intermediate region to conform with a first arc and a section of the inner surface of the intermediate region to conform with a second arc wherein the first arc has a radius of curvature r₁ around a point on a line perpendicular to the shaft at a midpoint between the proximal end and the distal end of the membrane and wherein the second arc has a radius of curvature r₂ around a point on the line, wherein r₁≧r₂ and the intermediate region of the membrane has a thickness t_{c} along the line;
affixing the proximal end of the balloon to an outer surface of the shaft and a distal end of the balloon to the outer surface of the shaft to establish an inflation chamber between the balloon membrane and the outer surface of the shaft; and
connecting an inflation unit in fluid communication with the inflation chamber of the balloon to inflate the balloon.

13. The method recited in claim 12 wherein the balloon membrane is made of a compliant material.

14. The method recited in claim 13 wherein the stretchability and bendability of the membrane material is directly proportioned to the thickness of the membrane.

15. The method recited in claim 13 wherein the distal region and the proximal region have a same, constant thickness t, and t is greater than t_{c}.

16. The method recited in claim 13 wherein the balloon membrane has a length L between the proximal and the distal end.

17. The method recited in claim 16 wherein L is less than 150 mm and the intermediate region corresponds with less than 90% of L.

18. The method recited in claim 16 wherein the intermediate region corresponds to 5% of L.

19. The method recited in claim 13 wherein the compliant material is urethane.

20. The method recited in claim 13 wherein during an inflation of the balloon, a radial distance r_{c} along the line from the longitudinal axis of the shaft to the outer surface of the intermediate region, is established by an inflation pressure Pᵢ inside the inflation chamber, wherein r_{c} varies proportionally with changes in Pᵢ inside the inflation chamber, and wherein the radial distance r_{c} is less than 35 mm and the inflation pressure Pᵢ is less than about 15 atmospheres.
